(19) United States
(12) Patent Application Publication
(10) Pub. No.: US 2015/0290105 A1
(43) Pub. Date: Oct. 15, 2015

(54) COMPOSITION COMPRISING SYRINGARESINOL FOR IMPROVING THE SKIN

(71) Applicant: AMOREPACIFIC CORPORATION, Seoul (KR)

(72) Inventors: Hyun Jung SHIN, Yongin-si (KR); Si Young CHO, Yongin-si (KR); Dae Bang SEO, Yongin-si (KR); Chan Woong PARK, Yongin-si (KR); Wan Gi KIM, Yongin-si (KR); Sang Jun LEE, Yongin-si (KR)

(21) Appl. No.: 14/353,711

(22) PCT Filed: Oct. 25, 2012
(86) PCT No.: PCT/KR2012/008816

§ 371 Date: Apr. 23, 2014

(30) Foreign Application Priority Data
Oct. 27, 2011 (KR) 10-2011-0110484
Nov. 1, 2011 (KR) 10-2011-0112764

(51) Int. Cl. A61K 8/49; A61Q 19/02; A61K 8/97; A61Q 19/08; A61Q 19/00

(52) U.S. Cl. CPC A61K 8/4973; A61Q 19/08; A61Q 19/007; A61K 8/97; A61Q 19/02

(57) ABSTRACT

The present invention relates to a composition for improving the skin, which comprises the compound of chemical formula 1, the derivatives thereof, or the pharmaceutically acceptable salts thereof as active ingredients.

### TECHNICAL FIELD

The present disclosure relates to a composition for improving skin, containing compound of Chemical Formula 1, a derivative thereof or a pharmaceutically acceptable salt thereof as an active ingredient.

### BACKGROUND ART

The color of human skin is determined by various factors including the activity of melanin-producing melanocytes, the distribution of blood vessels, the thickness of skin, the presence of pigments such as carotenoids, bilirubin, etc., or the like. Among them, the production of melanin by melanocytes mediated by many enzymes including tyrosinase is one of the most important factors that determine the skin color.

Melanin present in skin plays an important role of protecting against UV, etc. However, in excess, it is known to induce skin pigmentations such as melasma, freckle, lentigo, etc., accelerate skin aging and cause skin cancer.

In order to treat or alleviate skin pigmentation caused by hyperproduction of melanin, various substances exhibiting skin whitening effects, such as ascorbic acid, kojic acid, arbutin, hydroquinone, glutathione or derivatives thereof, have been used. But, they are limited in applications because of imperfect skin whitening effect, unguaranteed safety, difficulty in formulation as cosmetics and stability problem.

Skin is the part of the body which is the most vulnerable to UV because it is directly exposed to sunlight. Upon exposure to UV, the skin exhibits biological responses such as burn, pigmentation, DNA damage, change in connective tissues, and cancer, depending on the degree. Also, prolonged exposure to UV causes early skin aging, thereby leading to wrinkle formation, pigmentation, change in connective tissues, change in the thickness of the epidermis, and so forth.

The connective tissue of the skin mainly consists of collagen and elastin. Since collagen and elastin give elasticity to the skin, the skin is easily damaged and ages if they are weakened. Matrix metalloproteinases (MMPs) are enzymes that are involved in the breakdown of collagen. As the skin ages, the expression of MMPs increases and the increased MMPs break down the skin collagen. As this mechanism is repeated, the skin develops wrinkles and ages earlier. Transforming growth factor β (TGF-β) is a substance involved in the growth and differentiation of various cells. In mammals, TGF-β exists in three different isoforms. Among them, TGF-β1 exhibits the strongest activity for the formation of extracellular matrix (ECM) in the dermis and is involved in the restoration and differentiation of tissues. TGF-β1 produces various ECM components such as type I and type II collagen, fibronectin, proteoglycan, etc. by stimulating fibroblasts. Accordingly, a substance that facilitates the production of TGF-β1 may be able to prevent skin aging and improve skin wrinkles by promoting the growth of fibroblasts and accelerating skin regeneration.

To keep the skin moisturized, it is necessary to increase natural moisturizing factors in the skin. Since amino acids as the major component of the natural moisturizing factors are supplied from the breakdown of the protein filaggrin produced by keratinocytes, promoted filaggrin production may result in skin moisturizing effect. Also, since involucrin is a factor that plays a critical role in the formation of a cell envelope that prevents skin loss and the structuralization of the horny layer, promoted expression thereof may also lead to superior skin moisturizing effect.

### DISCLOSURE

### Technical Problem

The present disclosure is directed to providing a composition having excellent skin improving effect.

The present disclosure is also directed to providing a composition having excellent skin whitening effect and a composition having excellent antiaging effect.

The present disclosure is also directed to providing a composition having excellent skin aging preventing effect by preventing skin wrinkle formation, enhancing skin regeneration ability, promoting skin moisturization, etc. Also, the present disclosure is directed to providing an agent for improving or preventing wrinkles, an agent for promoting skin regeneration and an agent for moisturizing skin.

### Technical Solution

In an aspect, the present disclosure provides a composition for improving skin, containing a compound of Chemical Formula 1, a derivative thereof or a pharmaceutically acceptable salt thereof as an active ingredient:

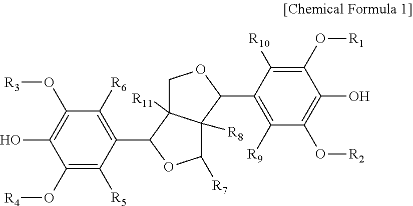

wherein

R₁, R₂, R₃ or R₄ is independently an unbranched or branched C₁-C₁₈ alkyl group, C₁-C₁₈ alkoxy group, C₁-C₁₈ alkenyl group, C₁-C₁₈ alkynyl group or C₃-C₆ cyclic alkyl group, and

R₅, R₆, R₇, R₈, R₉, R₁₀ or R₁₁ is independently hydrogen or an unbranched or branched, C₁-C₁₈ alkyl group, C₁-C₁₈ alkoxy group, C₁-C₁₈ alkenyl group, C₁-C₁₈ alkynyl group or C₃-C₆ cyclic alkyl group.

### Advantageous Effects

A composition according to the present disclosure, which contains a compound of Chemical Formula 1, specifically syringaresinol, as an active ingredient may exhibit excellent skin whitening effect by preventing melanin production, inhibiting tyrosinase activity and providing superior antioxidant effect and keratin removing ability.

The composition according to the present disclosure, which contains a compound of Chemical Formula 1, specifically syringaresinol, as an active ingredient may improve skin wrinkles by increasing the expression of SIRT1, suppressing the expression of MMP-9 induced by UV and inhibiting the breakdown of collagen which has an important effect on skin elasticity and strength. Also, it may exhibit superior skin cell regeneration promoting and skin elasticity enhancing effects by increasing the expression of TGF-β1 which promotes regeneration of cells. In addition, it may exhibit excellent skin moisturizing effect by promoting the expression of the skin moisturizing factors filaggrin and involucrin. Through these functions, the composition containing the compound of Chemical Formula 1, specifically syringaresinol, may exhibit superior effect of preventing aging, specifically skin aging, more specifically UV-induced skin aging.

### BRIEF DESCRIPTION OF DRAWINGS

FIG. 1 schematically describes a method of isolating and purifying syringaresinol from a ginseng berry extract.

FIG. 2 compares the free radical scavenging ability of syringaresinol with vitamin C.

FIG. 3 shows the SIRT1 expression promoting effect of syringaresinol in keratinocytes.

FIG. 4 shows the SIRT1 expression promoting effect of syringaresinol in fibroblasts.

FIG. 5 shows the filaggrin and involucrin expression promoting effect of syringaresinol.

FIG. 6 shows the MMP-9 expression and collagen breakdown inhibiting effect of syringaresinol.

FIG. 7 shows the skin wrinkle inhibiting effect of syringaresinol upon application or oral administration. FIG. 8 shows the transepidermal water loss (TEWL) inhibiting effect of syringaresinol upon application or oral administration.

### BEST MODE FOR CARRYING OUT INVENTION

In the present disclosure, the term “extract” is used as a broad concept and refers to any substance extracted from a natural product, regardless of extraction method, extraction solvent, extracted ingredients or the type of extract.

As used herein, the term “derivative” refers to any compound having substituent(s) at substitutable position(s) of the corresponding compound. The substituent is not particularly limited. For example, the substituent may independently be a C₁₋₁₀ acyclic hydrocarbon group which may be substituted with hydroxyl, phenoxy, thienyl, furyl, pyridyl, cyclohexyl, alkylalcohol, alkyldialcohol or substituted phenyl; a C₅₋₆ cyclic hydrocarbon group which may be substituted with hydroxyl, hydroxymethyl, methyl or amino; or a sugar residue, although not being limited thereto. As used herein, the term “sugar residue” refers to the group available on elimination of one hydrogen atom from a carbohydrate molecule. As such, it may mean, for example, a residue derived from a monosaccharide or an oligosaccharide.

As used herein, the term “pharmaceutically acceptable” means being devoid of substantial toxic effects when used with a usual medicinal dosage and thereby being approvable or approved by a regulatory agency of the government or being listed in the US Pharmacopoeia or other generalized recognized pharmacopoeia for use in animals, more particularly in human.

As used herein, the term “pharmaceutically acceptable salt” refers to a salt of the compound of the present disclosure which is pharmaceutically acceptable and possesses the desired pharmacological activity of the parent compound. The salt may include: (1) an acid addition salt formed with an inorganic acid such as hydrochloric acid, hydrobromic acid, sulfuric acid, nitric acid, phosphoric acid, etc. or formed with an organic acid such as acetic acid, propionic acid, hexanoic acid, cyclopentylpropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, ethane-1,2-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid or muconic acid; or (2) a salt formed when an acidic proton present in the parent compound is replaced. In addition to the pharmaceutically acceptable salt, the compound according to the present disclosure may include any salt, hydrate or solvate that can be prepared according to commonly employed methods.

As used herein, the term “skin” refers to a tissue that covers the body surface of an animal and is used in the broadest concept, including not only the tissue that covers the surface of face or body but also the scalp and hair.

As used herein, the term “improvement” refers to any positive effect on normal or abnormal skin, including moisturization, wrinkle improvement, etc.

Specifically, the composition of the present disclosure may provide skin improving effect by enhancing skin whitening or preventing aging, although not being limited thereto.

Hereinafter, the present disclosure is described in detail.

In an aspect, the present disclosure provides a composition for improving skin, containing a compound of Chemical Formula 1, a derivative thereof or a pharmaceutically acceptable salt thereof as an active ingredient:

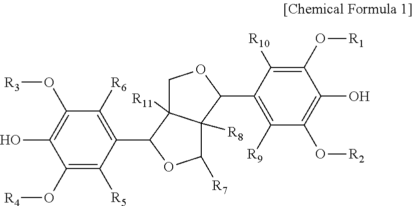

wherein

R₁, R₂, R₃ or R₄ is independently an unbranched or branched C₁-C₁₈ alkyl group, C₁-C₁₈ alkoxy group, C₁-C₁₈ alkenyl group, C₁-C₁₈ alkynyl group or C₃-C₆ cyclic alkyl group, and

R₅, R₆, R₇, R₈, R₉, R₁₀ or R₁₁ is independently hydrogen or an unbranched or branched, C₁-C₁₈ alkyl group, C₁-C₁₈ alkoxy group, C₁-C₁₈ alkenyl group, C₁-C₁₈ alkynyl group or C₃-C₆ cyclic alkyl group.

In an exemplary embodiment of the present disclosure, the compound may be syringaresinol.

As used herein, the term “syringaresinol” refers to a lignan-based compound having a chemical structure represented by Chemical Formula 2. It may be synthesized chemically or extracted from one or more of flax seed, phellodendri cortex, acanthopanacis cortex, sesame seed and ginseng berry. The flax seed, phellodendri cortex, acanthopanacis cortex and sesame seed respectively include all parts of the plant, for example, leaves, stem, root, fruit or seed and the ginseng berry includes the rind or pulp of ginseng berry.

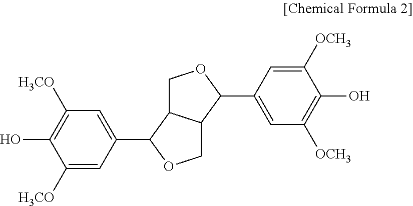 [SMILES: COc1cc(C2OCC3C(c4cc(OC)c(O)c(OC)c4)OCC23)cc(OC)c1O]

In the present disclosure, the “syringaresinol” may be obtained by extracting one or more of flax seed, phellodendri cortex, acanthopanacis cortex, sesame seed and ginseng berry with water, an organic solvent or a mixture of water and an organic solvent. The organic solvent includes one or more selected from a group consisting of alcohol, acetone, ether, ethyl acetate, diethyl ether, methyl ethyl ketone and chloroform, although not being limited thereto. The alcohol includes a C₁-C₅ lower alcohol and the C₁-C₅ lower alcohol includes one or more selected from a group consisting of methanol, ethanol, isopropyl alcohol, n-propyl alcohol, n-butanol and isobutanol, although not being limited thereto.

In an exemplary embodiment of the present disclosure, syringaresinol may be isolated and purified from ginseng berry by a procedure including: preparing an alcohol extract of ginseng berry pulp; eluting the prepared alcohol extract with a solvent including one or more of water and alcohol and obtaining fractions thereof; and performing chromatography, specifically thin-layer chromatography (TLC), on the obtained fractions using an organic solvent as an eluent. The organic solvent may include one or more selected from a group consisting of alcohol, acetone, ether, ethyl acetate, diethyl ether, methyl ethyl ketone and chloroform, and the alcohol may include a C₁-C₅ alcohol. In an exemplary embodiment of the present disclosure, the composition may contain the syringaresinol purified as described above as an active ingredient.

In an exemplary embodiment of the present disclosure, the composition may contain the active ingredient in an amount of 0.001-20 wt %, specifically 0.01-10 wt %, more specifically 0.1-5 wt %, based on the total weight of the composition. This range is appropriate not only to derive the effect desired by the present disclosure and satisfy both the stability and safety of the composition but also in terms of cost effectiveness. Specifically, if the content of the active ingredient is less than 0.01 wt %, sufficient skin whitening effect may not be achieved. And, if it exceeds 20 wt %, the safety and stability of the composition may be unsatisfactory.

In an exemplary embodiment of the present disclosure, the composition for improving skin whitens skin.

Skin hyperpigmentation such as melasma, freckle, lentigo, etc. may be caused by abnormality in melanocytes in skin and their surrounding environment. The inventors of the present disclosure have researched to develop a substance which is capable of reducing the production of melanin by improving melanocytes and their microenvironment, rapidly discharging produced melanin and exhibiting skin whitening effect by improving the brightness and uniformity of skin color. As a result, they have developed a composition for whitening skin, containing the compound of Chemical Formula 1, specifically syringaresinol, as an active ingredient.

The compound of Chemical Formula 1, specifically syringaresinol, may exhibit excellent skin whitening effect by reducing the toxicity of skin cells, specifically melanocytes, inhibiting production of melanin by melanocytes and inhibiting tyrosinase activity. In addition, it exhibits excellent effect of suppressing skin pigmentation such as tanning of the skin by sunlight by inhibiting oxidation in the microenvironment surrounding melanocytes with superior antioxidant effect and stabilizing the melanocytes. Also, it may provide healthy and bright skin color and skin tone and suppress hyperpigmentation by increasing the expression of interleukin 6 (IL-6) which is a cytokine excreted by keratinocytes, etc., brightens skin color and skin tone and suppresses skin hyperpigmentation such as melasma and blemish. Furthermore, since it has a superior effect of removing the skin keratin and facilitating keratin turnover, it may provide white and clean skin by removing the old keratin containing melanin. Also, the compound of Chemical Formula 1, specifically syringaresinol, may double the skin whitening effect by facilitating discharge of melanin and thereby making the skin surface smooth and soft. Accordingly, a composition according to the present disclosure containing the compound of Chemical Formula 1, specifically syringaresinol, as an active ingredient may have very superior skin whitening effect, specifically the effect of improving the brightness and uniformity of skin color, improving skin hyperpigmentation, removing keratin and facilitating and keratin turnover. The skin hyperpigmentation may include one or more selected from melasma, freckle, blemish, age spot, lentigo, birthmark and dark circle, although not being limited thereto.

In the present disclosure, improvement of the brightness and uniformity of skin color means that the brightness of the overall skin color is improved and the uniformity of the overall skin color is improved as blemish is decreased.

In another exemplary embodiment of the present disclosure, the syringaresinol may be contained in the composition as an extract of flax seed, phellodendri cortex, acanthopanacis cortex, sesame seed or ginseng berry.

Specifically, it may be contained in a fraction which is particularly effective for skin whitening.

In another aspect, the present disclosure provides a composition for external application to skin, which contains the above-described composition as an active ingredient. In another aspect, the present disclosure provides a composition for oral administration, which contains the above-described composition as an active ingredient. The composition containing the compound of Chemical Formula 1, specifically syringaresinol, as an active ingredient may exhibit superior skin whitening effect when applied to the skin or orally administered.

Sirtuin 1 (SIRT1) is a mammalian homolog of the silent information regulator 2 (Sir2) of yeast. It is an NAD⁺-dependent histone deacetylase. SIRT1 is known to be involved in resistance to aging and stress by regulating deacetylation of various proteins including histones, transcription factors, etc. It is also known that if SIRT1 is activated or overexpressed in human dermal cells and keratinocytes, the breakdown of collagen is prevented as the UV-induced MMP-9 expression is inhibited. This suggests that an agent that regulates the activation or expression of SIRT1 may suppress or prevent skin aging. However, no substance is available as yet which increases the expression of SIRT1 and has stability.

In an exemplary embodiment of the present disclosure, the composition for improving skin inhibits skin aging.

As used herein, the term aging refers to the degenerative change occurring as an organism grows older and includes decline in physiological activity, decreased metabolism and skin aging. The skin aging includes UV-induced wrinkle formation, decrease in moisturizing factors and loss of regenerating ability.

The compound of Chemical Formula 1, specifically syringaresinol, can increase the expression of SIRT1 4-fold or more, promote skin regeneration by increasing the expression of TGF-β1, which is indicative of skin regeneration ability, and suppress the UV-induced MMP-9 expression to a level as low as that in the absence of UV radiation. Also, the compound of Chemical Formula 1, specifically syringaresinol, may suppress the breakdown, specifically UV-induced breakdown, of collagen, which has an important effect on skin elasticity and strength. In addition, compound of Chemical Formula 1, specifically syringaresinol, may prevent skin dryness, prevent UV-induced skin barrier damage and quickly improve damaged skin barrier by promoting the expression of filaggrin and involucrin, which are important factors in skin moisturization, thereby keeping the skin surface moist. And, the compound of Chemical Formula 1, specifically syringaresinol, may suppress skin wrinkle formation and enhance skin elasticity by strengthening the connective tissues of skin. That is to say, the composition containing the compound of Chemical Formula 1, specifically syringaresinol, as an active ingredient may be used as an agent for improving or preventing wrinkles, an agent for promoting skin regeneration and an agent for moisturizing skin. As described above, since the compound of Chemical Formula 1, specifically syringaresinol, suppresses skin wrinkle formation, enhances skin regeneration ability and promotes skin moisturization, it exhibits excellent effect of preventing aging, specifically skin aging, more specifically UV-induced skin aging.

In another exemplary embodiment of the present disclosure, the syringaresinol may be contained in the composition as an extract of flax seed, phellodendri cortex, acanthopanacis cortex, sesame seed or ginseng berry. Specifically, it may be contained in a fraction which is particularly effective for preventing aging.

In another exemplary embodiment of the present disclosure, the composition may be a composition for external application to skin. In another aspect, exemplary embodiment of the present disclosure, the composition may be a composition for oral administration, containing syringaresinol as an active ingredient. The composition containing syringaresinol as an active ingredient may exhibit superior antiaging effect when applied on skin or orally administered.

In another exemplary embodiment of the present disclosure, the composition may be a cosmetic composition. The cosmetic composition may exhibit skin whitening effect and may specifically improve or prevent melasma, freckle, speckle, blemish or pigmentation on skin.

In another exemplary embodiment of the present disclosure, the composition may be a cosmetic composition containing syringaresinol as an active ingredient. The cosmetic composition may exhibit antiaging effect and, specifically, may prevent skin aging by suppressing skin wrinkle formation, enhancing skin regeneration ability, enhancing skin barrier function, promoting skin moisturization, or the like.

The cosmetic composition according to the present disclosure may be provided in the form of any formulation suitable for topical application. For example, it may be provided in the form of solution, oil-in-water emulsion, water-in-oil emulsion, suspension, solid, gel, powder, paste, foam or aerosol. These formulations may be prepared according to methods commonly employed in the art.

Specifically, the cosmetic composition according to the present disclosure may further contain other ingredients that may provide synergic effect to the desired main effect in a range not negatively affecting the main effect. Specifically, the cosmetic composition according to the present disclosure may further contain kojic acid, arbutin or ascorbic acid derivatives that may further enhance the skin whitening effect. Also, the cosmetic composition according to the present disclosure may further contain a humectant, an emollient, a surfactant, a UV absorbent, a preservative, a sterilizer, an antioxidant, a pH adjusting agent an organic or inorganic pigment, a fragrance, a cooling agent or a deodorant. The amount of these ingredients may be determined easily by those skilled in the art within a range not negatively affecting the purpose and effect of the present disclosure. They may be added in an amount of 0.01-5 wt %, specifically 0.01-3 wt %, based on the total weight of the composition.

In another exemplary embodiment of the present disclosure, the composition may be a pharmaceutical composition. The pharmaceutical composition may exhibit skin whitening effect and, specifically, may improve or prevent skin pigmentation such as melasma, freckle, freckle or blemish.

In another exemplary embodiment of the present disclosure, the composition may be a pharmaceutical composition exhibiting antiaging effect. Specifically, it may prevent skin aging by suppressing skin wrinkle formation, enhancing skin regeneration ability, enhancing skin barrier function, promoting skin moisturization, or the like.

In an exemplary embodiment of the present disclosure, the pharmaceutical composition may be administered orally or parenterally, e.g., rectally, topically, transdermally, intravenously, intramuscularly, intraperitoneally, subcutaneously, etc.

A formulation for oral administration may be tablet, pill, soft or hard capsule, granule, powder, fine granule, liquid, emulsion or pellet, although not being limited thereto. These formulations may further contain, in addition to the active ingredient, a diluent (e.g., lactose, dextrose, sucrose, mannitol, sorbitol, cellulose or glycine), a lubricant (e.g., silica, talc, stearic acid or polyethylene glycol) or a binder (e.g., magnesium aluminum silicate, starch paste, gelatin, tragacanth, methyl cellulose, sodium carboxymethyl cellulose or polyvinylpyrrolidone). In some cases, they may further contain a pharmaceutical additive such as a disintegrant, an absorbent, a colorant, a flavoring agent, a sweetener, etc. The tablet may be prepared according to the common mixing, granulation or coating method.

A formulation for parenteral administration may be injection, drop, lotion, ointment, gel, cream, suspension, emulsion, suppository, patch or spray, although not being limited thereto.

The dosage of the active ingredient of the pharmaceutical composition according to the present disclosure will vary depending on the age, sex and body weight of a subject, particular pathological condition and severity thereof, administration route or the discretion of a diagnoser. Determination of the dosage considering these factors is in the level of those skilled in the art. A daily dosage may be, for example, 0.1-100 mg/kg/day, more specifically 5-50 mg/kg/day, although not being limited thereto.

In another exemplary embodiment of the present disclosure, the composition may be a food composition. The food composition includes an indulgence food or health food composition.

The formulation of the food composition is not particularly limited. For example, it may be formulated into tablet, granule, powder, liquid such as drink, caramel, gel, bar, etc. Those skilled in the art may select and add the ingredients commonly used in the art to each formulation of the food composition without difficulty. In this case, a synergic effect may be achieved.

Determination of the dosage of the active ingredient is in the level of those skilled in the art. A daily dosage may be, for example, 0.1-5000 mg/kg/day, more specifically 50-500 mg/kg/day. However, the dosage may vary depending on various factors including the age and physical condition of a subject, the presence or absence of complication(s), or the like, without being limited thereto.

### Mode for Invention

Hereinafter, the present disclosure will be described in detail through an example and test examples. However, the following example and test examples are for illustrative purposes only and it will be apparent to those of ordinary skill in the art that the scope of the present disclosure is not limited by the example and test examples.

### EXAMPLE

### Isolation and Analysis of Syringaresinol

1. Pretreatment of Ginseng Berry

Live ginseng berry was harvested. After removing the seed and rind of ginseng berry, only the pulp was dried under sunlight or using hot air to obtain dried ginseng berry pulp.

2. Isolation of Syringaresinol from Ginseng Berry Pulp Extract and Analysis Thereof

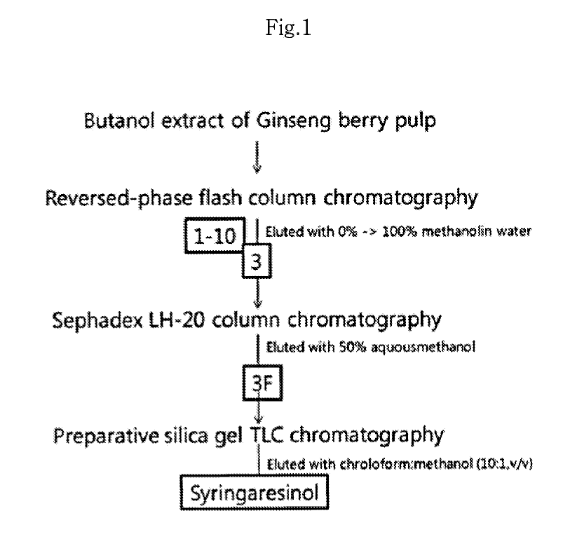

3 L of water or alcohol was added to 1 kg of the dried ginseng berry pulp. After extracting at room temperature or under reflux, followed by filtering and concentration at 40-45° C. under reduced pressure, 300 g of a ginseng berry pulp extract was obtained. The extract was treated with ether to remove oil-soluble components and then crude saponin was extracted with butanol and concentrated. Then, syringaresinol was isolated and purified therefrom as follows. First, 194 g of the sample was purified by reversed-phase (ODS) column chromatography. As the eluent, 100% water was used in the beginning. Subsequently, methanol was increased gradually by 10% and, finally, 100% methanol was used as the eluent. As a result, fractions GB-1 through GB-10 were obtained. Among the fractions, the fraction GB-3 which exhibits sirtuin 1 (SIRT1) expression activity was concentrated and subjected to Sephadex LH-20 column chromatography using 50% aqueous methanol. Among the resulting fractions, the fraction GB-3-6(3F) exhibiting SIRT1 expression activity was concentrated and subjected to preparative silica gel TLC using chloroform:methanol (10:1) as an eluent. As a result, an active fraction with an R_{f} value of 0.67 was purified. This procedure is schematically described in FIG. 1.

Through NMR spectroscopic analysis and database search, the isolated and purified active compound was identified as syringaresinol. Mass analysis was conducted to confirm this. As a result of ESI-mass analysis in the positive mode, [M+Na]⁺ (m/z=440.9) and [2M+Na]⁺ (m/z=858.9) peaks were observed and the molecular weight was calculated as 418. And, the result of NMR spectroscopic analysis was as in Chemical Formula 3. Accordingly, the isolated and purified active compound was confirmed to be syringaresinol.

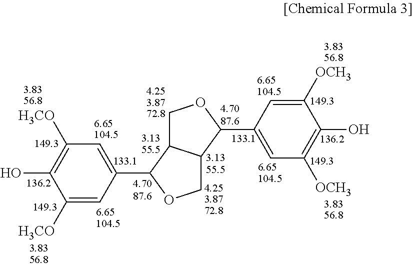 [SMILES: COc1cc(C2OCC3C(c4cc(OC)c(O)c(OC)c4)OCC23)cc(OC)c1O]

As such, syringaresinol was isolated from the ginseng berry pulp.

### Test Example 1

### Test of Cytotoxicity

Cytotoxicity was tested for syringaresinol in mouse melanocytes as follows.

Melanocytes (Mel-Ab cells) derived from C57BL/6 (treated with Dooley et al.'s method) were cultured under the condition of 37° C. and 5% CO₂ in Dulbecco's modified Eagle's medium (DMEM) containing 10% fetal bovine serum, 100 nM 12-O-tetradecanoylphorbol-13-acetate and 1 nM cholera toxin. The cultured Mel-Ab cells were detached with 0.25% trypsin-EDTA, cultured in a 24-well plate to a concentration of 10⁵ cells/well and then treated with the syringaresinol prepared in Example. After culturing for 24 hours, the cells were washed with phosphate buffered saline (PBS, pH 7.2) and then stained using 0.1% crystal violet (Sigma) and 10% ethanol (EtOH) for 5 minutes at room temperature. After completely removing free crystal violet, the cells were washed 3 times with PBS (pH 7.2). The stained cells were treated with 95% ethanol (EtOH) for 1 hour at room temperature to extract crystal violet. The absorbance of the extract was measured at 540 nm using the Lambda 25 spectrophotometer (PerkinElmer) and cell viability (%) was calculated according to Equation 1. The result is shown in Table 1.

Cell viability (%)=(A/B)×100 [Equation 1]

A: absorbance in the presence of syringaresinol

B: absorbance in the absence of syringaresinol

TABLE 1

| Test substance | Cell viability (%) |
|---|---|
| Syringaresinol | 98.6 |

As seen from Table 1, syringaresinol can increase cell viability by significantly reducing the toxicity of skin cells.

### Test Example 2

### Evaluation of Melanin Production Inhibiting Effect

The melanin production inhibiting effect of the syringaresinol obtained in Example was evaluated using mouse melanocytes.

First, melanocytes (Mel-Ab cells) derived from C57BL/6 (treated with Dooley et al.'s method) were cultured under the condition of 37° C. and 5% CO₂ in Dulbecco's modified Eagle's medium (DMEM) containing 10% fetal bovine serum, 100 nM 12-O-tetradecanoylphorbol-13-acetate and 1 nM cholera toxin. The cultured Mel-Ab cells were detached with 0.25% trypsin-EDTA, cultured in a 24-well plate to a concentration of 10⁵ cells/well and then treated with 10 ppm hydroquinone as a positive control or the syringaresinol prepared in Example for 3 consecutive days from day 2. After removing the culture medium and washing with PBS, the cells were dissolved with 1 N sodium hydroxide and absorbance was measured at 400 nm. Melanin production inhibiting rate (%) was calculated according to Equation 2 from the measured absorbance. The result is shown in Table 2.

Melanin production inhibiting rate (%)=100−(Absorbance of test substance) /(Absorbance of control substance)×100 [Equation 2]

TABLE 2

| Test substance | Melanin production inhibiting rate (%) |
|---|---|
| Syringaresinol | 85.7 |
| Hydroquinone (positive control) | 58.9 |

As seen from Table 2, syringaresinol exhibits superior melanin production inhibiting rate (%) and the effect is much better than that of the positive control hydroquinone. Accordingly, it can be seen that syringaresinol can provide superior skin whitening effect by inhibiting melanin production.

### Test Example 3

### Evaluation of Tyrosinase Activity Inhibiting Effect

The following experiment was carried out to investigate whether syringaresinol inhibits the activity of tyrosinase which is the most important enzyme in melanin synthesis.

A mixture of 0.1 M melanin production inhibiting rate buffer (pH 6.8), 0.03% L-tyrosine solution (0.3 mg/mL in D.W.) and 1.42 units/μL tyrosinase solution (PBS) was used to prepare a reaction solution. The reaction solution was prepared by adding 500 μL of 0.1 M PBS, 15 μg of L-tyrosine and 50 μL of syringaresinol and then adding distilled water to make the final volume 1500 μL. A solution prepared through the same procedure but with Kojic acid instead of syringaresinol was used as a positive control. The concentration of all the samples used in experiment was set to 10 ppm. After adding 10 units of mushroom-derived tyrosinase to the reaction solution, followed by reaction at 37° C. for 10 minutes, the reaction was stopped by quickly transferring onto ice for 5 minutes. The synthesis rate of dopachrome produced was determined by measuring absorbance at 475 nm and tyrosinase activity inhibiting rate (%) was calculated therefrom. The result is shown in Table 3.

TABLE 3

| Test substance | Tyrosinase activity inhibiting rate (%) |
|---|---|
| Syringaresinol | 90.2 |
| Kojic acid (positive control) | 67.8 |

As seen from Table 3, syringaresinol exhibits superior tyrosinase activity inhibiting effect. Accordingly, it can be seen that syringaresinol can provide superior skin whitening effect by inhibiting melanin production by melanocytes.

### Test Example 4

### Measurement of Free Radical Scavenging Ability

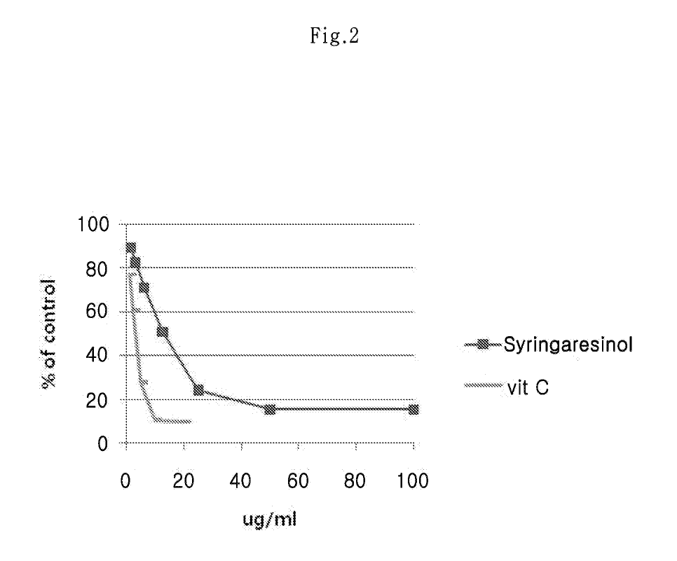

A radical solution was prepared by dissolving 100 M diphenylpicrylhydrazyl (DPPH), composed of stable free radicals, in 99% ethanol. A reaction solution was prepared by dissolving syringaresinol in distilled water with different concentrations (pg/mL) and mixing the syringaresinol solution with the radical solution. A reaction solution not containing syringaresinol was used as a negative control and a reaction solution containing vitamin C (Vit C) was used as a positive control. After sufficiently reacting at 37° C. for 30 minutes, radical scavenging ability was evaluated by measuring absorbance at 515 nm. The radical scavenging ability was calculated relative to the negative control group as 100. The result is shown in FIG. 2.

As seen from FIG. 2, syringaresinol can scavenge free radicals in a concentration-dependent manner and the effect is superior to that of vitamin C. Accordingly, it can be seen that syringaresinol has very superior antioxidant effect.

### Test Example 5

### Evaluation of Interleukin 6 Expression Increasing Effect

Interleukin 6 (IL-6) is a cytokine secreted by keratinocytes, etc. Increased secretion owing to activation of the IL-6 gene leads to bright skin color and skin tone and suppressed skin pigmentation such as melasma or blemish. It was investigated whether syringaresinol can increase the expression of the IL-6 gene in skin keratinocytes.

Keratinocytes were treated with 10 ppm or 100 ppm syringaresinol. 24 hours later, the cells were recovered and washed twice with 10 mL of PBS. Then, total RNA was isolated from the cells using TRIzol reagent (Invitrogen, Carlsbad, Calif., USA). The isolated RNA was purified once more using the Qiagen RNeasy kit (Qiagen, Valencia, Calif.) and cDNA was synthesized therefrom using the Superscript Reverse Transcriptase (RT) II kit (Invitrogen, Carlsbad, Calif.). Subsequently, the change in the expression of the interleukin 6 gene was quantitatively analyzed by real-time reverse transcription polymerase chain reaction (Q-RT-PCR). The change in the gene expression pattern was evaluated using the TaqMan gene expression assay kit (Applied Biosystems, Forster City, Calif.). Hs00174360_ml was used as a primer. The expression level of interleukin 6 in the cells was analyzed by real-time PCR and was calculated relative to a non-treated control group. The result is shown in Table 4.

TABLE 4

| Test substance | Expression of IL-6 gene |
|---|---|
| Control (none) | 1 |
| Syringaresinol (10 ppm) | 3.5 |
| Syringaresinol (100 ppm) | 8.2 |

As seen from Table 4, syringaresinol can increase the expression of interleukin 6 in keratinocytes in a concentration-dependent manner. This means that syringaresinol can induce less production of melanin by secreting interleukin 6 to melanocytes, thereby exhibiting superior skin whitening effect.

### Test Example 6

### Evaluation of Skin Whitening Effect when Applied to Skin

The following experiment was carried out to investigate the human skin whitening effect of syringaresinol.

First, opaque tapes having holes of 1.5 cm in diameter were attached on the upper arms of 12 healthy male subjects and UVB of about 1.5-2 times the minimal erythemal dose was irradiated to each subject to induce skin tanning. After the UV radiation, 1% syringaresinol prepared in Example (1,3-butylene glycol:ethanol=7:3 was used as solvent), 1% hydroquinone (positive control) or 1% solvent (vehicle, negative control) was applied on one arm as test substances and nothing was applied on the other arm. Then, the change was monitored for 10 weeks. The color of the skin was measured with 1-week interval using the CR2002 colorimeter (Minolta, Japan). The change in skin color (ΔL*) between the time when the test substance was applied and the time when the test was completed was calculated according to Equation 3. The result is shown in Table 5. Skin whitening effect is evaluated by comparing the ΔL* of the part where the test substance was applied with that of the control part. A ΔL* value of about 2 can be interpreted as distinct skin whitening effect and one about 1.5 can be evaluated as positive skin whitening effect.

ΔL*=(L* when test was completed)−(L* when test substance was applied) [Equation 3]

TABLE 5

| Test substance | Change in skin brightness (ΔL*) |
|---|---|
| Syringaresinol | 1.98 ± 0.11 |
| Hydroquinone (positive control) | 1.71 ± 0.31 |
| Solvent (vehicle, negative control) | 0.53 ± 0.25 |

As seen from Table 5, syringaresinol exhibits superior skin brightness not only to the negative control but also to the positive control hydroquinone. Accordingly, it can be seen that syringaresinol exhibits superior skin whitening effect by suppressing death of skin cells and improving UV-induced skin pigmentation.

### Test Example 7

### Evaluation of Skin Color Uniformity when Applied to Skin

The following experiment was carried out to investigate the human skin color uniformity enhancing effect of syringaresinol.

Test substances were applied on the skin of the subjects in substantially the same manner as in Test Example 6. Skin color uniformity was evaluated as follows. Skin brightness of randomly selected three skin parts was measured and standard deviation thereof was calculated. A smaller standard deviation was evaluated as better skin brightness uniformity. The measurement was made using the same apparatus and method as in Test Example 6.

TABLE 6

| Test substance | Differences of Standard deviation |
|---|---|
| Syringaresinol | 0.08 |
| Hydroquinone (positive control) | 0.10 |
| Solvent (vehicle, negative control) | 0.27 |

As seen from Table 6, syringaresinol exhibits superior skin color uniformity not only to the negative control but also to the positive control hydroquinone. Accordingly, it can be seen that syringaresinol has superior effect of uniformly improving skin color.

### Test Example 8

### Evaluation of Keratin Reducing Effect

In order to investigate whether syringaresinol can reduce the keratin of skin, syringaresinol of the same concentration as in Test Example 6 was applied on the inside of the lower arms of 50 male and female adults in their 20s and 30s and without skin diseases. 24 hours later, the reduced amount of keratin was measuring using Charm View (Moritex, Japan). The initial amount of skin keratin prior to application of the test substance measured with Charm View under a constant-temperature (24° C.), constant-humidity (40%) condition was used as a reference value, and the change in the amount of keratin was measured 24 hours later. The result is shown in Table 7.

TABLE 7

| Test substance | Before application | 24 hours later |
|---|---|---|
| Syringaresinol | 20.3 | 11.9 |
| Control | 20.8 | 21.4 |

As seen from Table 7, syringaresinol has remarkably superior effect of reducing skin keratin. Accordingly, it can be seen that syringaresinol can provide superior skin whitening effect by removing melanin-containing old keratin.

### Test Example 9

### Evaluation of Skin Whitening Effect and Skin Color Uniformity when Orally Administered

Brown guinea pig was used as an animal model to investigate whether syringaresinol exhibits skin whitening effect when orally administered. Minimal erythemal dose was measured after irradiating UV to guinea pigs. After grouping the guinea pigs with 10 per each group, UV of the minimal erythemal dose was irradiated once a day, for a total of 3 times. The guinea pigs were allowed free access to feed for 5 weeks. The feed contained 1% syringaresinol or 1% vitamin C (Vit C) as a positive control. Skin pigmentation was measure using a colorimeter in substantially the same manner as in Test Example 6. The result is shown in Table 8.

TABLE 8

| Test substance | Skin color brightness (ΔL*) |
|---|---|
| Syringaresinol | 1.67 ± 0.25 |
| Vitamin C (positive control) | 1.48 ± 0.71 |
| Normal feed (negative control) | 0.43 ± 0.33 |

As seen from Table 8, syringaresinol exhibits superior skin whitening effect to vitamin C when orally administered. This means that syringaresinol can provide superior skin whitening effect not only when applied on skin but also when orally administered.

Also, skin color uniformity was evaluated in substantially the same manner as in Test Example 7. The result is shown in Table 9.

TABLE 9

| Test substance | Differences of Standard deviation |
|---|---|
| Syringaresinol | 0.07 |
| Vitamin C (positive control) | 0.11 |
| Normal feed (negative control) | 0.32 |

As seen from Table 9, syringaresinol exhibits superior skin color uniformity not only to the negative control but also to the positive control vitamin C. Accordingly, it can be seen that syringaresinol has superior effect of uniformly improving skin color when orally administered.

### Test Example 10

### Evaluation of SIRT1 Expression Promoting Effect in Human Keratinocytes and Fibroblasts

The following experiment was carried out to evaluate the SIRT1 gene expression promoting effect of syringaresinol in human keratinocytes and fibroblasts.

1. Cell Lines and Cell Culturing

Human keratinocyte HaCaT cells or human fibroblast HDF cells were cultured under the condition of 37° C. and 5% CO₂ in DMEM ((Gibco 1210-0038)) containing 10% fetal bovine serum.

2. Evaluation of SIRT1 Expression Promoting Effect

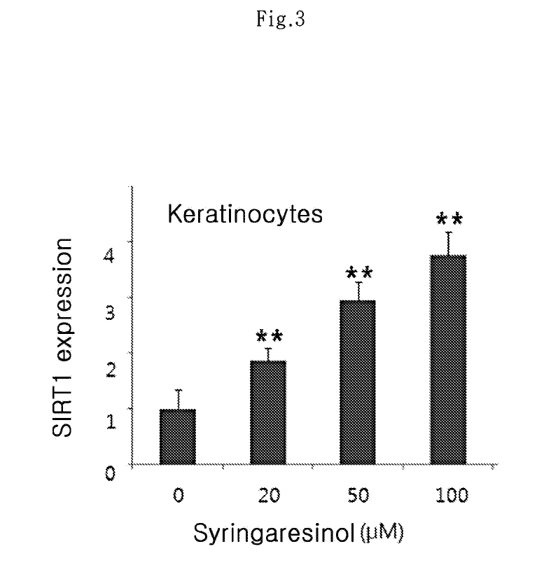

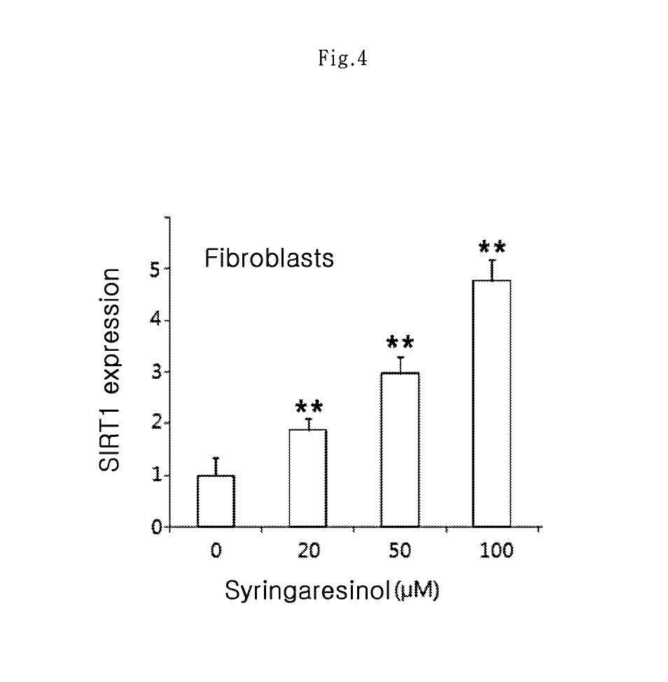

The cultured cells were treated with syringaresinol dissolved in DMSO at a concentration of 20, 50 or 100 μM for 24 hours. Separately from this, the cells were treated with DMSO of 1/1000 of the volume of the culture medium as a negative control. After treating with the test substance, the cells were washed twice with cold PBS and RNA was extracted using TRIzol reagent (Invitrogen). Then, cDNA was synthesized using 1 μg/μL of the extracted RNA and a reverse transcription system (Promega). The expression pattern of the SIRT1 and GAPDH genes was monitored using the synthesized cDNA and predesigned primers and probes (Applied Biosystems; SIRT1, Hs01009006_ml; GAPDH, Hs99999905_ml). PCR reaction and analysis were carried out using the Rotor-Gene 3000 system (Corbett Research, Sydney, Australia). The result is shown in FIG. 3 (keratinocytes) and FIG. 4 (fibroblasts).

As seen from FIG. 3 and FIG. 4, syringaresinol can increase the expression of SIRT1 in human keratinocytes and fibroblast by 4 times or more in a concentration-dependent manner.

### Test Example 11

### Evaluation of Filaggrin and Involucrin Expression Promoting Effect in Human Keratinocytes

The following experiment was carried out to evaluate the filaggrin and involucrin expression promoting effect of syringaresinol in human keratinocytes.

1. Cell Line and Cell Culturing

Human keratinocyte HaCaT cells were cultured in substantially the same manner as in Test Example 10.

2. Evaluation of Filaggrin and Involucrin Expression Promoting Effect

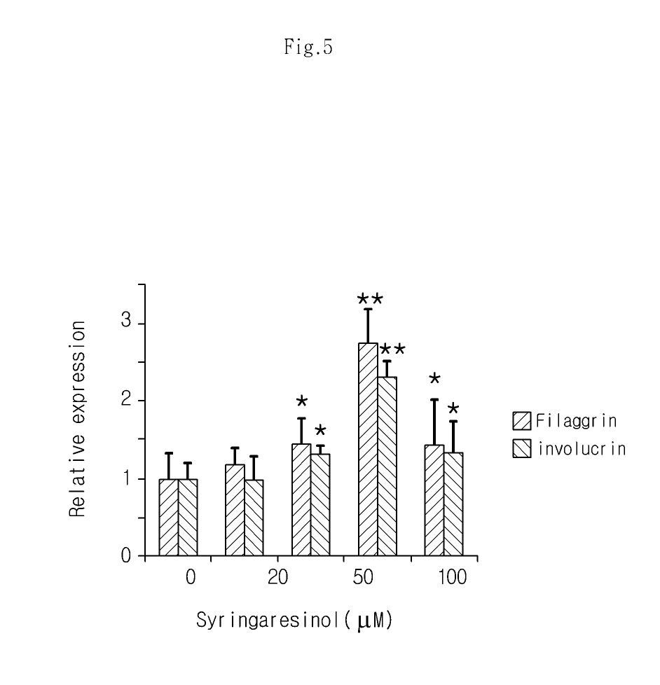

cDNA was synthesized in substantially the same manner as in Test Example 10 and the expression pattern of the filaggrin, involucrin and GAPDH genes was monitored substantially the same manner as in Test Example 10 using the synthesized cDNA and predesigned primers and probes (Applied Biosystems; SIRT1, Hs01009006_ml; GAPDH, Hs99999905_ml). The result is shown in FIG. 5.

As seen from FIG. 5, syringaresinol increases the expression of filaggrin and involucrin in human keratinocytes in a concentration-dependent manner. Accordingly, it can be seen that syringaresinol enhances skin barrier function and exhibits skin moisturizing effect.

### Test Example 12

### Evaluation of MMP-9 Expression and Collagen Breakdown Inhibiting Effect in Human Keratinocytes

The following experiment was carried out to evaluate the MMP-9 expression and collagen breakdown inhibiting effect of syringaresinol in human keratinocytes.

1. Cell Line and Cell Culturing

Human keratinocyte HaCaT cells were cultured in substantially the same manner as in Test Example 10.

2. Evaluation of UV-Induced MMP-9 Expression and Collagen Breakdown Inhibiting Effect

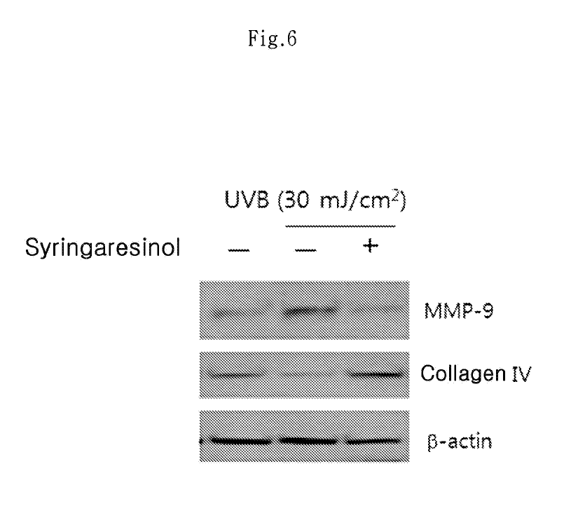

The cultured cells were treated with trypsin to obtain a single-cell suspension. Then, the cells were distributed in a 6-well plate, with 2×10⁵ cells per well, and cultured for 24 hours. Subsequently, after replacing the medium with DMEM not containing fetal bovine serum and culturing again for 24 hours, the cells were treated with 50 μM syringaresinol dissolved in DMSO. After treating for 24 hours, the cells were washed with PBS and UVB of 30 mJ/cm² was irradiated in PBS. After discarding the PBS, the cells were added to a medium containing syringaresinol of the same concentration. As a control group, the cells were cultured in the same manner without treating with syringaresinol. 24 hours after the UV radiation, the cells treated and untreated with syringaresinol were washed with PBS, recovered by treating with trypsin, treated with 500 mL of protein extraction buffer (8 M urea, 2% CHAPS, 50 mM DTT, 2 M thiourea, 2 mM PMSF, 100 mg/mL leupeptin) and kept at room temperature for 10 minutes. Then, after centrifuging at 4° C. for 10 minutes with 15,000 g, the supernatant was recovered and protein was quantitated using Bio-Rad Protein Dye ReagentTM. 20 mg of protein was fractionated by size using 8% SDS-PAGE and blotted on PDF membrane (Bio-Rad) for 12 hours with 50 V. The resulting blot was blocked for 1 hour with 5% non-fat milk and reacted using monoclonal anti-MMP-9 antibody (Santa Cruz, Calif., USA) and monoclonal anti-collagen IV antibody (Abcam, Cambridge, Mass., USA) as primary antibodies, horse radish peroxidase-conjugated anti-mouse IgG (Amersham) as secondary antibody and the enhanced chemiluminescence (ECL) kit (Amersham). The reacted blot was exposed to X-ray Fuji film and developed to investigate the protein expression level. Bands on the film were scanned with PowerLook 2100 XL (UMAX) and analyzed with the image-analyzing program ImageMaster 2D Elite (Amersham Bioscience). The result is shown in FIG. 6.

As seen from FIG. 6, UV radiation leads to increased MMP-9 expression and decreased collagen as a result of facilitated collagen breakdown in keratinocytes. However, treatment with syringaresinol inhibits collagen breakdown by inhibiting the UV-induced increase of MMP-9 expression, thereby preventing UV-induced decrease in collagen production. That is to say, syringaresinol can prevent and improve skin wrinkles by inhibiting MMP-9 expression and collagen breakdown and thus can prevent skin aging.

### Test Example 13

### Evaluation of TGF-β1 Expression Promoting Effect in Fibroblasts

The following experiment was carried out to evaluate the TGF-β1 expression promoting effect of syringaresinol in fibroblasts.

1. Cell Line and Cell Culturing

Human fibroblast HDF cells were cultured in substantially the same manner as in Test Example 10.

2. Evaluation of TGF-β1 Expression Promoting Effect

The cultured cells were treated with syringaresinol dissolved in DMSO to a concentration of 20, 50 or 100 μM for 24 hours. After 24 hours, the cell culture was recovered.

The quantity of TGF-β1 produced in the recovered cell culture was measured using the Quantikine™ high-sensitivity ELISA kit (R&D Systems, USA) according to the manufacturer's instructions. All the samples required for the measurement of the quantity of TGF-β1 were provided by the kit. First, the recovered cell culture was transferred to a 96-well microplate uniformly coated with TGF-β1 antibody and it was allowed for antigen-antibody reaction to proceed at 37° C. for 1 hour. After removing the cell culture, the wells were washed 3 times with PBS. Then, horseradish peroxidase-conjugated secondary antibody was added to each well and it was allowed for antigen-antibody reaction to proceed for 1 hour. Subsequently, after adding tetramethylbenzidine as a chromogenic substrate to each well, followed by reaction at room temperature for 15 minutes, 1 N sulfuric acid was added to stop the reaction. Then, absorbance was measured at 450 nm using a spectrophotometer. After drawing a standard curve using a standard solution, the TGF-β1 production amount in the cell culture with the test substance added was calculated from the measured absorbance. The result is shown in Table 10.

TABLE 10

| Syringaresinol (μM) | TGF-β1 |
|---|---|
| 0 | 23.7 ± 1.2 |
| 20 | 43.3 ± 1.0 |
| 50 | 87.2 ± 4.2 |
| 100 | 92.1 ± 5.6 |

As seen from Table 10, syringaresinol increases the production of TGF-β1 in fibroblasts in a concentration-dependent manner. Accordingly, it can be seen that syringaresinol can promote skin regeneration by increasing TGF-β1 production and thus can prevent skin aging and improve skin wrinkles.

### Test Example 14

### Evaluation of Wrinkle Formation and Transepidermal Water Loss Inhibiting Effect in Hairless Mouse Skin

The following experiment was carried out to evaluate the wrinkle formation and transepidermal water loss inhibiting effect of syringaresinol.

1. Treatment of Hairless Mouse

30-week-old female hairless mice (SKH: HR-1) weighing 25-39 g were purchased from Charles River Laboratories (Wilmington, Mass., USA). The mice were accustomed to the laboratory environment for a week and then grouped into normal groups and test substance-treated groups, with 5 mice per each group. The mice were allowed free access to water and feed under the condition of 12-hour light/dark cycles, 23.2° C. and 55.10% humidity.

2. Evaluation of Skin Wrinkle Formation Inhibiting Effect of Syringaresinol when Applied on Skin or Orally Administered

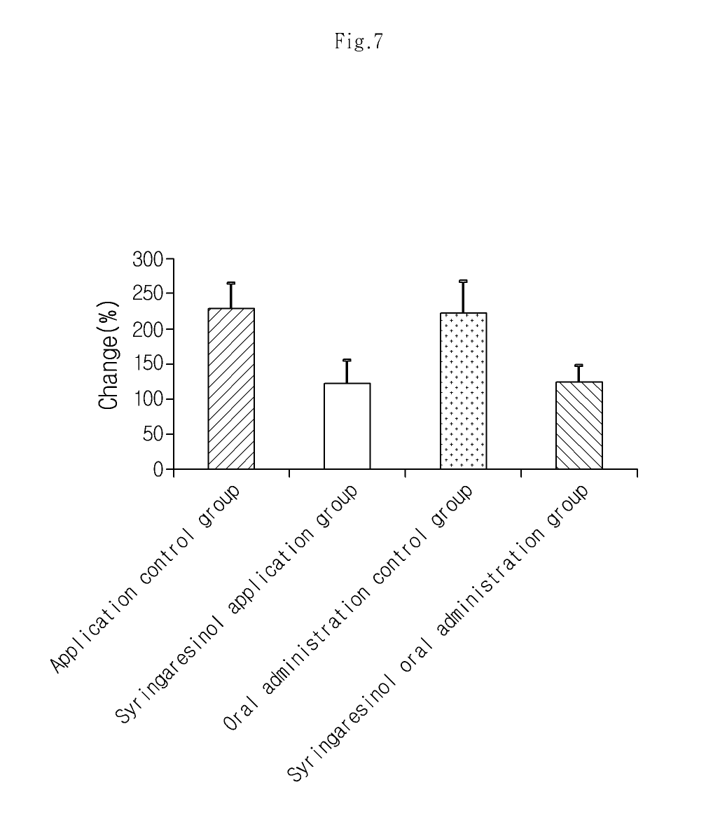

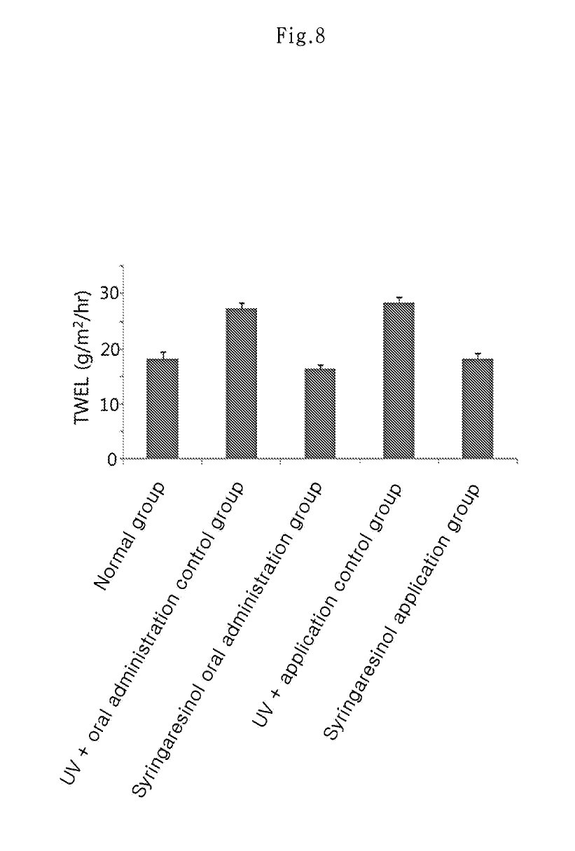

For the test substance-treated groups, a patch of 1% syringaresinol dissolved in a solvent (1,3-butylene glycol:ethanol=7:3) was applied 2 times for 2 days or 200 mg/kg syringaresinol was orally administered. The control groups were treated in the same way using the solvent of the same amount instead of syringaresinol. The syringaresinol-applied group, syringaresinol oral administration group and control groups were exposed to UVB of 180 mJ/cm². Before and after the UV radiation, skin wrinkles on the exposed part were taken with replica and measured using the Visiometer system (C+K). The change in skin wrinkles was calculated according to Equation 4 and the result is shown in FIG. 7.

Change (%)=[(T_{di}−T_{do})/(T_{do})]×100 [Equation 4]

T_{di}: value measured after UV radiation

T_{do}: value measured before UV radiation

As seen from FIG. 7, the application and oral administration control groups show increase of skin wrinkles by 230±35% and 224±45%, respectively, whereas the syringaresinol application and oral administration groups show only slight increase of 121±35% and 123±25%, respectively. Accordingly, it can be seen that syringaresinol has very superior skin wrinkle inhibiting effect.

3. Measurement of Transepidermal Water Loss (TEWL)

3 weeks after the onset of experiment, transepidermal water loss (TEWL) was measured on the back part of the hairless mouse of each group using a wireless vapometer (Delfin Technologies, Ltd., SWL 4102). The result is shown in FIG. 8. The measurement environment was 60±5% relative humidity and 25.5±0.5° C.

Transepidermal water loss is the quantity of water that passes from inside the body through the skin and a larger value means poor skin moisturization ability and damaged skin barrier function. Accordingly, the transepidermal water loss may be used as an index in evaluating skin barrier function, recovery of damaged skin barrier function, protection of skin from stimulations such as UV, and so forth.

As seen from FIG. 8, UV radiation results in deteriorated skin moisturization ability because of damaged skin barrier function. Oral administration or application of syringaresinol leads to decrease in UV-induced transepidermal water loss. Accordingly, it can be seen that syringaresinol can provide superior skin barrier function recovering, skin protecting and skin moisturizing effect by preventing UV-induced transepidermal water loss.

Hereinafter, the present disclosure will be described in detail through formulation examples. However, the formulation examples are for illustrative purposes only and it will be apparent to those of ordinary skill in the art that the scope of the present disclosure is not limited by the formulation examples.

### Formulation Example 1

### Lotion

A lotion is prepared according to a commonly employed method with the following composition.

TABLE 11

| Ingredients | Content (wt %) |
|---|---|
| Syringaresinol | 3.0 |
| L-Ascorbic acid-2-phosphate magnesium salt | 1.0 |
| Water-soluble collagen (1% aqueous solution) | 1.0 |
| Sodium citrate | 0.1 |
| Citric acid | 0.05 |
| 1,3-Butylene glycol | 3.0 |
| Purified water | balance |

### Formulation Example 2

### Cream

A cream is prepared according to a commonly employed method with the following composition.

TABLE 12

| Ingredients | Content (wt %) |
|---|---|
| Syringaresinol | 1.0 |
| Polyethylene glycol monostearate | 2.0 |
| Glycerin monostearate | 5.0 |
| Cetyl alcohol | 4.0 |
| Squalene | 3.0 |
| Glyceryl tri(2-ehtylhexanoate) | 6.0 |
| Sphingolipid | 1.0 |
| 1,3-Butylene glycol | 7.0 |
| Purified water | balance |

### Formulation Example 3

### Pack

A pack is prepared according to a commonly employed method with the following composition.

TABLE 13

| Ingredients | Content (wt %) |
|---|---|
| Syringaresinol | 5.0 |
| Polyvinyl alcohol | 13.0 |
| L-Ascorbic acid-2-phosphate magnesium salt | 1.0 |
| Lauroyl hydroxyproline | 1.0 |
| Water-soluble collagen (1% aqueous solution) | 2.0 |
| 1,3-Butylene glycol | 3.0 |
| Ethanol | 5.0 |
| Purified water | balance |

### Formulation Example 4

### Soft Capsule

After mixing 100 mg of syringaresinol, 50 mg of soybean extract, 180 mg of soybean oil, 50 mg of red ginseng extract, 2 mg of palm oil, 8 mg of hydrogenated palm oil, 4 mg of yellow beeswax and 6 mg lecithin, a soft capsule is prepared according to a commonly employed method.

### Formulation Example 5

### Tablet

Granules formed by mixing 100 mg of syringaresinol g, 50 mg of soybean extract, 100 mg of glucose, 50 mg of red ginseng extract, 96 mg of starch and 4 mg of magnesium stearate and adding 40 mg of 30% ethanol are dried and prepared into a tablet.

### Formulation Example 6

### Health Food

| | | |
|---|---|---|
| Syringaresinol | 1000 | mg |
| Vitamin mixture | | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Mineral mixture | | |
| Ferrous sulfate | 1.75 | mg |
| Zinc oxide | 0.82 | mg |
| Magnesium carbonate | 25.3 | mg |
| Potassium dihydrogen phosphate | 15 | mg |
| Calcium monohydrogen phosphate | 55 | mg |
| Potassium citrate | 90 | mg |
| Calcium carbonate | 100 | mg |
| Magnesium chloride | 24.8 | mg |

Although the above-described mixing ratios of the vitamin and mineral mixtures are provided as specific examples suitable for health food, the mixing ratios may be changed as desired.

### Formulation Example 7

### Health Drink

| | | |
|---|---|---|
| Syringaresinol | 1000 | mg |
| Citric acid | 1000 | mg |
| Oligosaccharide | 100 | g |
| Taurine | 1 | g |

| | |
|---|---|
| Purified water | balance |

According to a commonly employed method, the above-described ingredients are mixed and stirred for about 1 hour while heating at about 85° C. The resulting solution is filtered and sterilized.

### INDUSTRIAL APPLICABILITY

A composition according to the present disclosure has superior antioxidant effect and keratin removing ability and thus can exhibit excellent skin whitening effect.

The composition according to the present disclosure may provide superior effect of preventing aging, specifically skin aging, more specifically UV-induced skin aging.

1. A method for improving skin in a subject, the method comprising administering an effective amount of a compound of Chemical Formula 1, a derivative thereof or a pharmaceutically acceptable salt thereof to the subject: 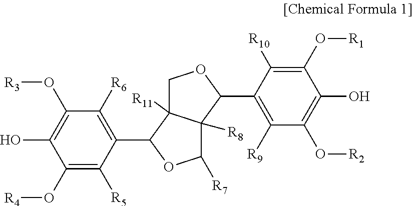
wherein
R₁, R₂, R₃ or R₄ is independently an unbranched or branched C₁-C₁₈ alkyl group, C₁-C₁₈ alkoxy group, C₁-C₁₈ alkenyl group, C₁-C₁₈ alkynyl group or C₃-C₆ cyclic alkyl group, and
R₅, R₆, R₇, R₈, R₉, R₁₀ or R₁₁ is independently hydrogen or an unbranched or branched, C₁-C₁₈ alkyl group, C₁-C₁₈ alkoxy group, C₁-C₁₈ alkenyl group, C₁-C₁₈ alkynyl group or C₃-C₆ cyclic alkyl group.

2. The method according to claim 1, wherein the compound is syringaresinol.

3. The method according to claim 2, wherein the syringaresinol is included in an extract of one or more selected from flax seed, phellodendri cortex, acanthopanacis cortex, sesame seed and ginseng berry.

4. The composition according to claim 2, wherein the syringaresinol whitens skin.

5. The method according to claim 4, wherein the syringaresinol improves the brightness and uniformity of skin color.

6. The method according to claim 4, wherein the syringaresinol improves skin hyperpigmentation.

7. The method according to claim 6, wherein the skin hyperpigmentation comprises one or more selected from melasma, freckle, blemish, age spot, lentigo, birthmark and dark circle.

8. The method according to claim 2, wherein the syringaresinol facilitates keratin removal and keratin turnover.

9. The method according to claim 2, wherein the syringaresinol inhibits skin aging.

10. The method according to claim 2, wherein the syringaresinol improves or inhibits skin wrinkles.

11. The method according to claim 2, wherein the syringaresinol promotes skin regeneration.

12. The method according to claim 2, wherein the syringaresinol promotes skin moisturization.

13. The method according to claim 2, wherein the syringaresinol is administered in a form of is a cosmetic composition.

14. The method according to claim 2, wherein the syringaresinol is administered in a form of a pharmaceutical composition.

15. The method according to claim 13, wherein the composition comprises 0.001-20 wt % of the syringaresinol based on the total weight of the composition.

16. The method according to claim 14, wherein the composition comprises 0.001-20 wt % of the syringaresinol based on the total weight of the composition.
